(19) United States
(12) Patent Application Publication
(10) Pub. No.: US 2013/0171037 A1
(43) Pub. Date: Jul. 4, 2013

(54) DENTAL TOOL STERILIZER

(71) Applicant: Doo Man Im, Gwangju-si (KR)

(72) Inventor: Doo Man Im, Gwangju-si (KR)

(73) Assignee: SEMANTECH CO., LTD., Gwangju-si (KR)

(21) Appl. No.: 13/057,524

(22) PCT Filed: Dec. 3, 2010
(86) PCT No.: PCT/KR2010/008623

§ 371 Date: Feb. 4, 2011

(30) Foreign Application Priority Data
Sep. 9, 2010 (KR) 10-2010-0088437

(51) Int. Cl. A61L 2/10

(52) U.S. Cl. CPC A61L 2/10
USPC 422/186.3; 250/455.11

(57) ABSTRACT

Disclosed herein is a dental tool sterilizer, which sterilizes a dental tool to keep the dental tool sanitary and allows a dental tool that has been sterilized or is to be sterilized to be conveniently and easily put into and taken out of the sterilizer. The sterilizer includes a support body having a turntable on an upper portion thereof, a power button being provided at a predetermined position on the support body to control supply of power. A dental-tool protective casing is provided such that an open bottom thereof is placed on the upper portion of the support body, and has a door on a side thereof. A sterilizing means is provided in the casing and has an ultraviolet lamp that radiates ultraviolet rays. A dental-tool holding means is provided on the turntable in such a way as to rotate along with the turntable, and holds dental tools.

### TECHNICAL FIELD

The present invention relates to a dental tool sterilizer, which sterilizes various tools and side materials (hereinafter referred to as a “dental tool”) for a dental examination, treatment or operation, thus keeping the dental tool sanitary and allows a dental tool that has been sterilized or is to be sterilized to be conveniently and easily put into and taken out of the sterilizer.

### BACKGROUND ART

Generally, among dental tools used is a bur that forms a hole in the mouth or cuts teeth to treat odontopathy. The bur is coupled to a handpiece to be operated. A cutting piece made of diamond or the like is coupled to an end of a shank having a predetermined length, thus providing a cutting part. Further, a file is inserted into the tooth to be used in an endodontic treatment, and is coupled to a handpiece or held by a dentist's hand when it is used in the treatment.

Since various kinds of burs and files are used according to the size and section to be treated, the dentist prepares a plurality of burs and files suitable to what is being treated. As such, since there are various kinds of burs and files, a stand is used to store the burs and files. Generally, the stand includes a support body which is provided with a plurality of insert holes to receive the burs and files, and a cover which is coupled to the support body to allow the burs and files to be selectively exposed to the outside.

However, the conventional stand is problematic in that it is not easy to place the burs or files according to size, and a space for separately storing the burs or files which have been used is not provided. Further, the conventional stand is problematic in that there is no partition, so that neighboring burs or files may be in friction contact with each other and thus be damaged when the burs or files are placed.

In order to solve these problems, Korean patent No. 10-0940602 has been proposed which was filed by the inventor of this invention and is entitled a rotary stand for dental tools. However, the rotary stand is problematic in that it is difficult to maneuver the dental tools into or out of the stand, it is very complicated in construction, and the dental tools are not normally sterilized so that it is impossible to keep the dental tools sanitary.

### DISCLOSURE

### Technical Problem

Accordingly, the present invention has been made keeping in mind the above problems occurring in the prior art, and an object of the present invention is to provide a dental tool sterilizer, which automatically sterilizes various kinds of dental tools for a dental examination, treatment or operation using an ultraviolet lamp, thus keeping the dental tools sanitary and preventing the transmission of germs, and which allows the dental tools to be conveniently and easily put into or taken out of the sterilizer, and in which the ultraviolet lamp for sterilizing the dental tools is designed to be harmless to humans, thus preventing a person from being affected by ultraviolet rays when a dental tool which is to be sterilized or has been sterilized is put into or taken out of the sterilizer.

### Technical Solution

In order to accomplish the above object, the present invention provides a dental tool sterilizer including a support body having a rotatable turntable on an upper portion thereof, a power button being provided at a predetermined position on the support body to control supply of power; a dental-tool protective casing provided such that an open bottom thereof is placed on the upper portion of the support body, and having a door on a side thereof; a sterilizing means provided in the dental-tool protective casing, and having an ultraviolet lamp that radiates ultraviolet rays when power is supplied to the sterilizing means; and a dental-tool holding means provided on the turntable in such a way as to rotate along with the turntable, and holding a plurality of dental tools.

The power button, timer up/down buttons, a display part, an ultraviolet-lamp operating time setting button, and a display lamp may be provided on a surface of the support body. The door may be a sliding door that conforms to a shape of the side of the dental-tool protective casing. A storage part may be provided on a central portion on an upper portion of the dental-tool protective casing to temporarily store a dental tool which is being used. The sterilizing means may include an ultraviolet lamp which is turned on or off in conjunction with operation of the power button. A sensor may be provided at a position adjacent to the door to control supply of power to the ultraviolet lamp.

According to an aspect of the present invention, the dental-tool holding means may include support columns erected vertically and spaced apart from each other by predetermined intervals, a support ring provided on each of upper and lower ends of the support columns, a plurality of holders provided on each of the support columns or the support ring and having a plurality of steps, and a dental-tool holding ring provided on each of the steps and having a dental-tool holding hole to hold each of the dental tools. A portion of the dental-tool holding ring may be cut to be open.

According to another aspect of the present invention, the dental-tool holding means may include support columns erected vertically and spaced apart from each other by predetermined intervals, a support ring provided on each of upper and lower ends of the support columns, and a plurality of holders provided on each of the support columns or the support ring. Each of the holders may include a plurality of steps, a light radiating space formed through a side surface of the holder, and a dental-tool holding hole formed in each of the steps in such a way as to communicate with the light radiating space. The dental-tool holding means may include a locking protrusion which protrudes upwards so as to make each of the dental tools be locked to a surface of the turntable in a standing position.

According to a further aspect of the present invention, the dental-tool holding means may include support columns erected vertically and spaced apart from each other by predetermined intervals, a support ring provided on each of upper and lower ends of the support columns, a tray support provided on each of the support columns or the support ring and having a locking step, and a tray coupled to the locking step and receiving the dental tools. A portion of the tray is cut to be open.

According to another aspect of the present invention, the dental-tool holding means may include a locking protrusion which protrudes upwards so as to make each of the dental tools be locked to a surface of the turntable in a standing position.

Further, an ultraviolet lamp may be provided on a surface of the turntable. A light reflecting film may be provided on an inner surface of each of the protective casing and the door. A surface of the light reflecting film may be coated with titanium dioxide.

### Advantageous Effects

As is apparent from the above description, a dental tool sterilizer according to the present invention is advantageous in that it automatically sterilizes various kinds of dental tools for a dental examination, treatment or operation using an ultraviolet lamp, thus keeping the dental tools sanitary and preventing the transmission of germs, and it allows the dental tools to be conveniently and easily put into or taken out of the sterilizer, and the ultraviolet lamp for sterilizing the dental tools is designed to be harmless to humans, thus preventing a person from being affected by ultraviolet rays when a dental tool which is to be sterilized or has been sterilized is put into or taken out of the sterilizer.

### DESCRIPTION OF DRAWINGS

FIG. 1 is a perspective view showing a dental tool sterilizer according to a first embodiment of the present invention, when a door is open;

FIG. 2 is an exploded perspective view showing the dental tool sterilizer according to the first embodiment of the present invention;

FIG. 3 is a perspective view showing a dental-tool holding means of the dental tool sterilizer according to the first embodiment of the present invention;

FIG. 4 is a perspective view showing a dental tool sterilizer according to a second embodiment of the present invention;

FIG. 5 is a perspective view showing a dental-tool holding means of the dental tool sterilizer according to the second embodiment of the present invention;

FIG. 6 is a perspective view showing a dental tool sterilizer according to a third embodiment of the present invention;

FIG. 7 is a perspective view showing a dental-tool holding means of the dental tool sterilizer according to the third embodiment of the present invention;

FIG. 8 is a perspective view showing a dental tool sterilizer according to a fourth embodiment of the present invention; and

FIG. 9 is a perspective view showing a dental-tool holding means of the dental tool sterilizer according to the fourth embodiment of the present invention.

### BEST MODE

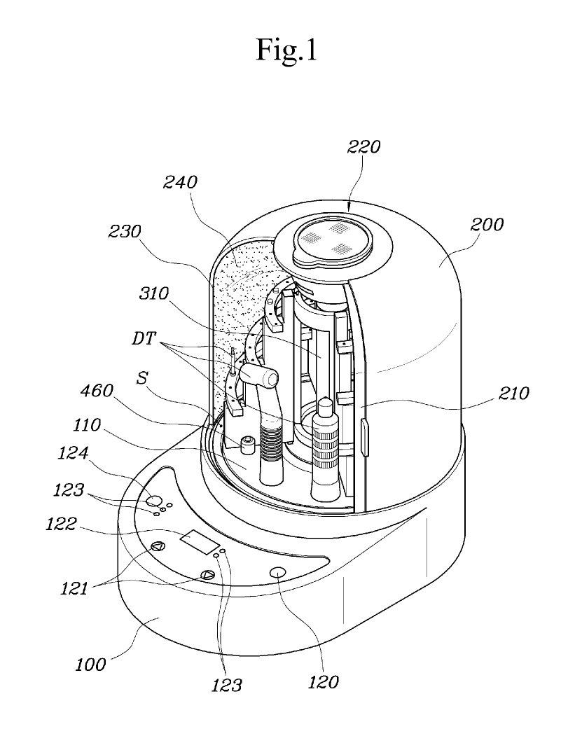

As shown in FIG. 1, a dental tool sterilizer according to the present invention includes a support body 100 and a dental-tool protective casing 200. The support body 100 is placed on a desired location, and includes a turntable 110 which is rotatably provided on an upper portion of the support body 100, and a power button 120 which is provided at a predetermined position on a surface of the support body 100 to control the supply of power. The dental-tool protective casing 200 is coupled at an open bottom thereof to the upper portion of the support body 100, with a door 210 provided on a side of the dental-tool protective casing 200.

Further, a sterilizing means 300 and a dental-tool holding means 400 are provided in the dental-tool protective casing 200. The sterilizing means 300 includes an ultraviolet lamp 310 that radiates ultraviolet rays when power is supplied to the sterilizing means 300. The dental-tool holding means 400 is placed on the turntable 110 in such a way as to rotate along with the turntable 110, and functions to hold a plurality of dental tools DT.

The dental-tool holding means 400 holds the dental tools DT to receive a maximum amount of ultraviolet rays which are radiated from the ultraviolet lamp 310. It is preferable that the ultraviolet lamp 310 be located along a central axis of the dental-tool protective casing 200. The turntable 110 is preferably rotated along with the dental-tool holding means 400 only when a person artificially rotates the turntable 110.

The door 210 is used to put or take the dental tools DT into or out of the dental-tool holding means 400. The door 210 is designed to be electrically operated such that the ultraviolet lamp 310 is turned off when the door 210 starts to be open, and the ultraviolet lamp 310 is turned on when the door 210 is closed.

A means for turning on or off the ultraviolet lamp 310 as the door 210 is opened or closed comprises a sensor S which is installed at a position adjacent to the door 210. If the sensor S comes into contact with an end of the door 210, the ultraviolet lamp 310 is turned on. Meanwhile, if the sensor S is spaced apart from the entrance of the door 210, the ultraviolet lamp 310 is turned off.

Thus, when the door 210 is closed and thus the ultraviolet lamp 310 is turned on, the dental tools DT held by the dental-tool holding means 400 are sterilized.

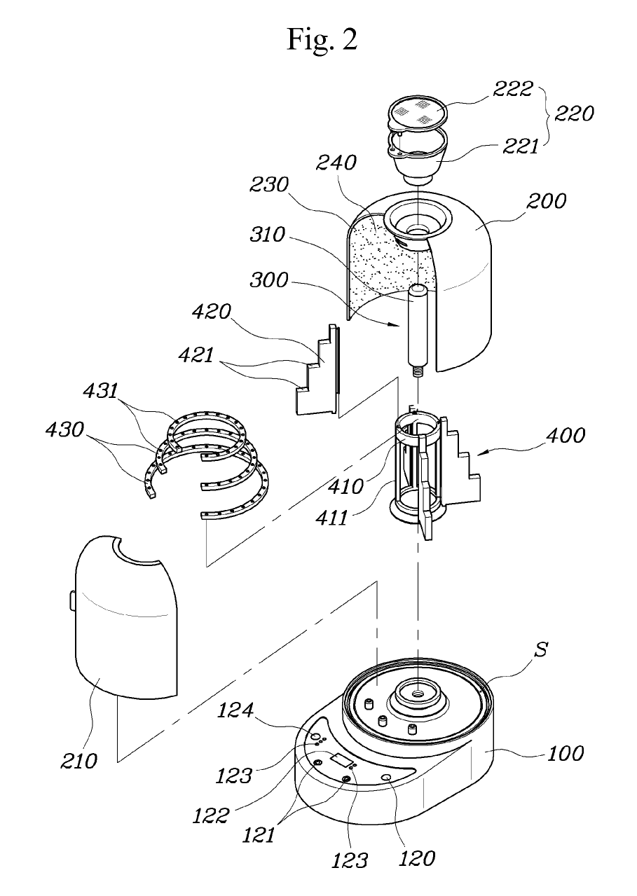

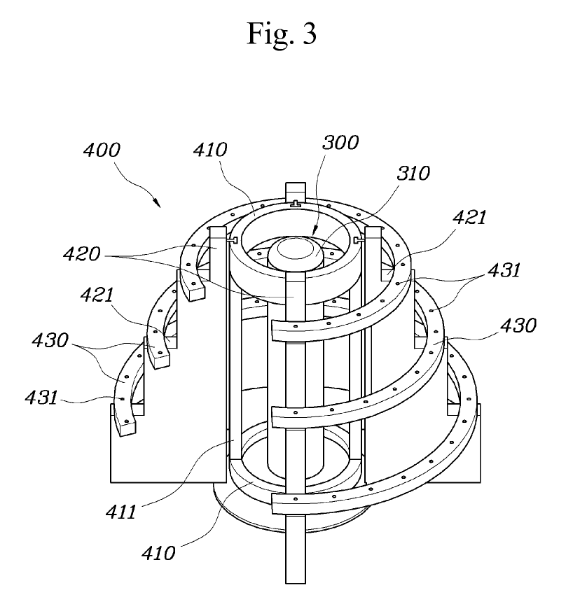

Hereinafter, a dental tool sterilizer according to a first embodiment of the present invention will be described in detail with reference to FIGS. 2 and 3.

The dental tool sterilizer according to the first embodiment of the present invention includes a support body 100 which is set in a desired location. A rotatable turntable 110 is provided on an upper portion of the support body 100. A power button 120 is provided at a predetermined position on a surface of the support body 100 to control the supply of power. In addition to the power button 120, there are timer up/down buttons 121, an ultraviolet-lamp operating time setting button 124, a display part 122, and display lamps 123 displaying whether the power button 120, the timer up/down buttons 121, and the display part 122 are operating normally or not.

The power button 120 is used to supply or cut off power to the ultraviolet lamp 310. The timer up/down buttons 121 are used to regulate lighting time of the ultraviolet lamp 310. The ultraviolet-lamp operating time setting button 124 is used to turn on the ultraviolet lamp 310 for a predetermined time and then to turn off the ultraviolet lamp 310. The display part 122 functions to display, in figures, the lighting time of the ultraviolet lamp 310 which is regulated by the timer up/down buttons 121. The display lamps 123 display whether the respective parts are operated or not.

Further, the turntable 110 rotates around an axis along which the ultraviolet lamp 310 is installed. The rotation of the turntable 110 is performed artificially by a person. Meanwhile, the power may use either or both of a battery and electricity.

The ultraviolet lamp 310 is provided on the central portion of the upper portion of the support body 100, and is electrically connected to the power button 120 to be turned on or off by the operation of the power button 120, so that the dental tools DT are sterilized by ultraviolet rays radiated from the ultraviolet lamp 310.

It is preferable that the sterilizing means 300 extend from the central portion of the upper portion of the support body 100 to the central portion of the upper portion of a dental-tool protective casing 200 which will be described below.

Further, a bottom of the dental-tool protective casing 200 is placed on an outer circumference of the turntable 110, in other words, the upper portion of the support body 100. The bottom of the dental-tool protective casing 200 is open, and the interior of the dental-tool protective casing 200 defines a space.

A storage part 220 is provided on the central portion of the upper portion of the dental-tool protective casing 200 in such a way as to be exposed to the outside, and functions to temporarily store the dental tools DT which are being used. Thus, the dental tools DT which are being used in a dental treatment or examination can be placed or stored in the storage part 220 for a time. The storage part 220 includes a funnel-shaped cup 221, and a cover 222 that covers a top of the cup 221. Thus, when a user wishes to use the storage part 220, the cover 222 is opened and then the dental tools DT are put into the cup 221. Preferably, the cover 222 may be made of fiber or non-woven fabric to allow the dental tools DT to be stuck into the cover 222.

Further, a dental-tool holding means 400 is provided in the dental-tool protective casing 200 to hold various kinds of dental tools DT which are sterilized by the ultraviolet rays radiated from the ultraviolet lamp 310. The detailed construction of the dental-tool holding means 400 will be described with reference to FIG. 3.

The dental-tool holding means 400 includes support columns 411 which are erected vertically in such a way as to be spaced apart from each other by predetermined intervals. Support rings 410 are provided on upper and lower ends of the support columns 411 to be supported by the support columns 411. A plurality of holders 420 is coupled to an outer surface of each support ring 410, and is provided with a plurality of steps 421. Dental-tool holding rings 430 are provided on the respective steps 421 and have dental-tool holding holes 431 to hold the dental tools DT.

In the dental-tool holding means 400 of the first embodiment constructed as described above, the plurality of dental-tool holding holes 431 are formed at predetermined intervals through each dental-tool holding ring 430. Thus, a dental tool DT which is to be sterilized is inserted and seated into an associated dental-tool holding hole 431. Here, the dental tool DT may be inserted into the dental-tool holding hole 431 such that a lower end of the dental tool DT faces downwards or upwards.

The dental-tool holding rings 430 holding the dental tool DT as such are seated in stages on the holders 420 having the steps 421. The holders 420 are detachably mounted to the support rings 410 and the support columns 411. The plurality of steps 421 formed on the holders 420 allow a plurality of dental-tool holding rings 430 to be seated thereon.

Further, a portion of each dental-tool holding ring 430 is cut to be open, so that the dental tool DT can be locked in the open portion in a standing position. In order to lock the dental tool DT to a predetermined position, locking protrusions 460 are provided on a surface of the turntable 110 in such a way as to protrude upwards, and function to lock the dental tools DT in the standing position. A lower portion of each dental tool DT is fitted over a corresponding locking protrusion 460 to be locked. A dental tool DT which may be fitted over the locking protrusion 460 is a handpiece, etc.

The above description pertains to the dental tool sterilizer according to the first embodiment of the present invention, while the following description pertains to a dental tool sterilizer according to a second embodiment of the present invention.

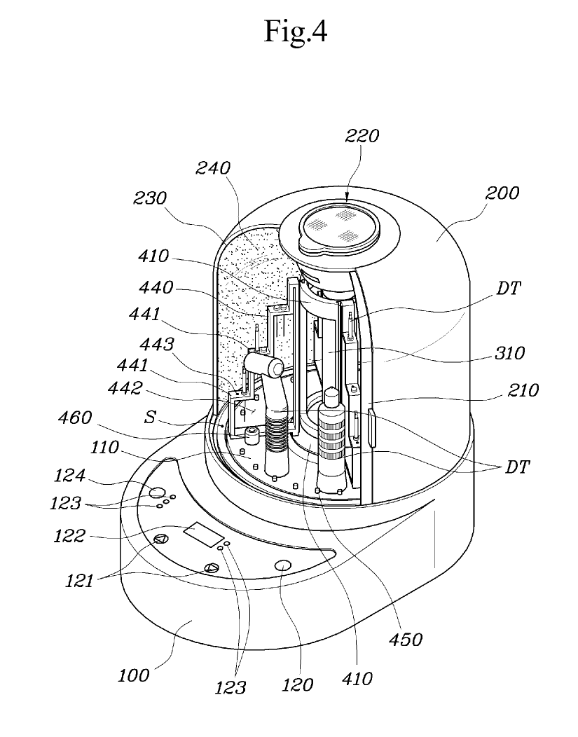

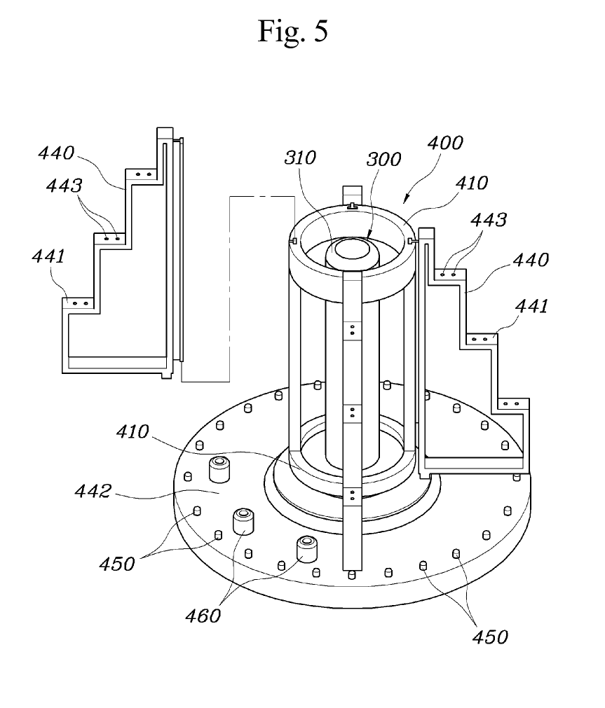

As shown in FIGS. 4 and 5, the dental tool sterilizer according to the second embodiment of the present invention includes a support body 100, a dental-tool protective casing 200, and a sterilizing means 300. The support body 100 includes a turntable 110 which is rotatably provided on an upper portion of the support body 100, and a power button 120 which is provided at a predetermined position on a surface of the support body 100 to control the supply of power. The dental-tool protective casing 200 is coupled at an open bottom thereof to the upper portion of the support body 100, with a door 210 provided on a side of the dental-tool protective casing 200. The sterilizing means 300 is provided in the dental-tool protective casing 200, and includes an ultraviolet lamp 310 that radiates ultraviolet rays when power is supplied to the sterilizing means 300. Since the above components are equal to those of the first embodiment, they will not be described below. A dental-tool holding means 400 of the second embodiment different from that of the first embodiment will be described below in detail.

The dental-tool holding means 400, according to the second embodiment of the present invention, includes support columns 411 which are erected vertically in such a way as to be spaced apart from each other by predetermined intervals. Support rings 410 are provided on upper and lower ends of the support columns 411. A plurality of holders 440 is coupled to each support ring 410 or support column 411. Each holder 440 is provided with a plurality of steps 441, and is open at a side to form a light radiating space 442, with dental-tool holding holes 443 being formed in a surface of each step 441 and communicating with the light radiating space 442. In the dental-tool holding means 400 of the second embodiment constructed as described above, a dental tool in question (a dental tool which is to be sterilized) is inserted and seated into the dental-tool holding hole 443 which is formed in the surface of the step 441 in such a way as to communicate with the light radiating space 442. The dental tool DT may be inserted and seated into the dental-tool holding hole 443 such that a lower end of the dental tool DT faces downwards or upwards.

The dental tool 412 seated into the dental-tool holding hole 443 as such is sterilized by ultraviolet rays radiated from the ultraviolet lamp 310. The steps 441 formed on the holder 440 allows a larger number of dental tools DT to be seated, in addition to adjusting positions in which dental tools DT having different lengths are seated.

Further, ultraviolet lamps 450 may be provided at predetermined intervals along the outer circumference of the turntable 110. Locking protrusions 460 may be provided on a surface of the turntable 110 in such a way as to protrude upwards, thus making the dental tools DT be locked in a standing position. A lower portion of each dental tool DT is fitted over the corresponding locking protrusion 460. A dental tool DT which may be fitted over the locking protrusion 460 is a handpiece, etc.

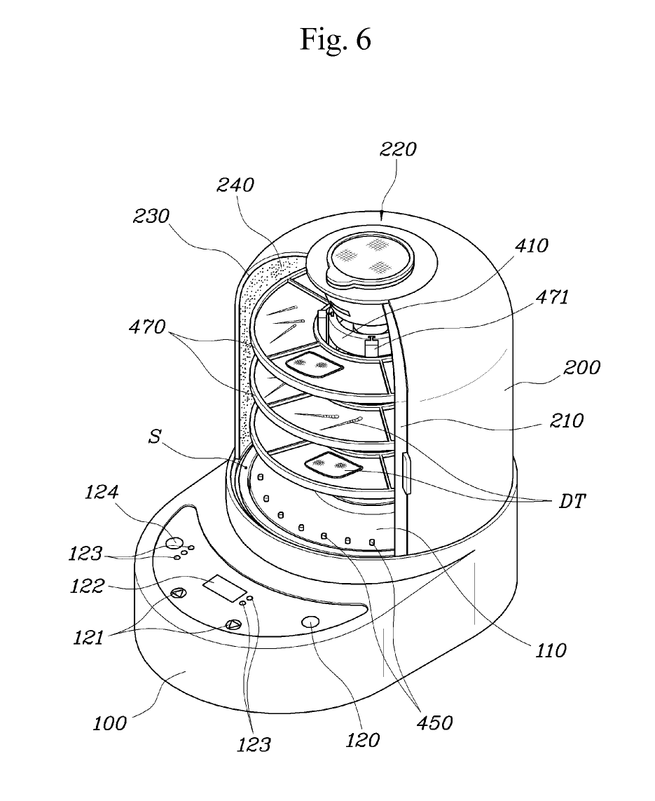

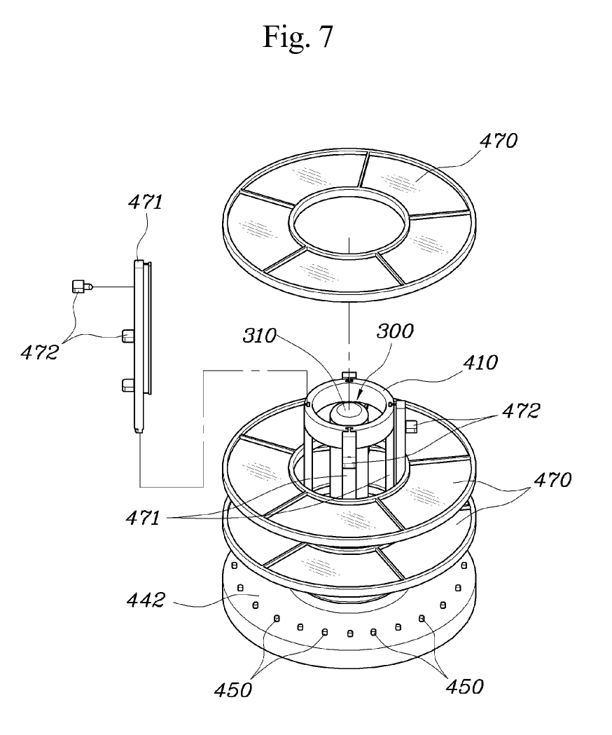

As shown in FIGS. 6 and 7, a dental-tool holding means 400 according to a third embodiment of the present invention includes support columns 411 which are erected vertically in such a way as to be spaced apart from each other by predetermined intervals. Support rings 410 are provided on upper and lower ends of the support columns 411. Tray supports 471 having locking steps 472 are coupled to the respective support rings 410 or support columns 411. A tray 470, receiving dental tools DT, is coupled to the locking steps 472.

The tray 470 is a part in which the dental tools DT, that is, various tools or side materials (gauze, GP cone, gutta-percha point, paper point, etc.) used in a dental examination, treatment, or operation, are received and then sterilized using ultraviolet rays radiated from the ultraviolet lamp 310. The tray 470 has the shape of a plate. Partitions are provided at predetermined intervals in the tray 470 to divide the receiving space into several portions.

Further, the tray 470 may be cut at a portion thereof to be open.

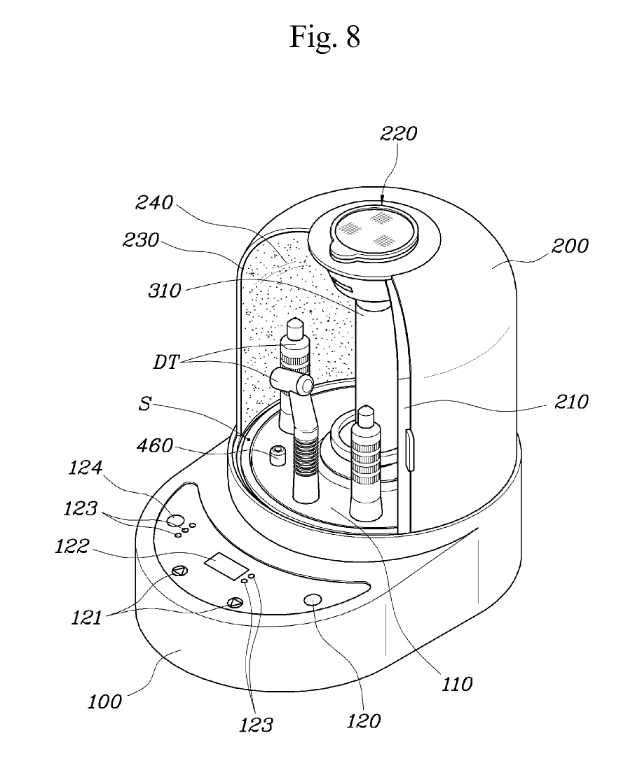

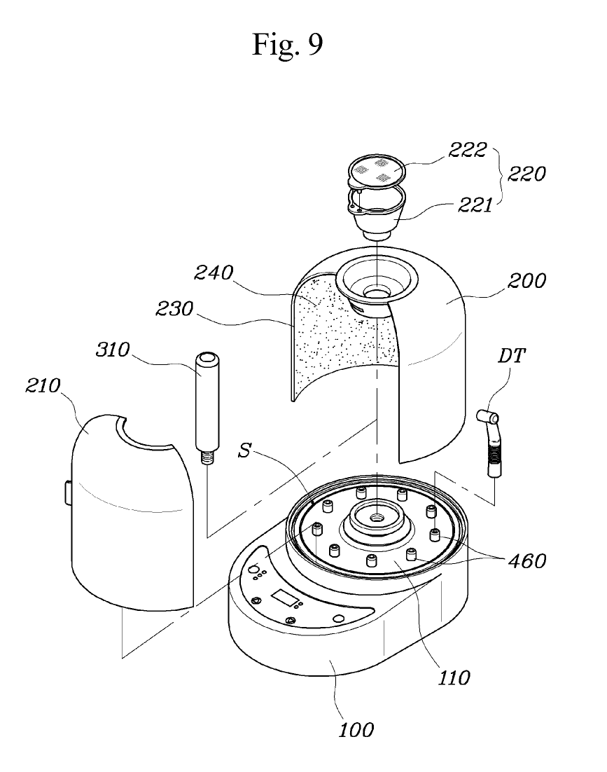

As shown in FIGS. 8 and 9, a dental-tool holding means 400 according to a fourth embodiment of the present invention includes a support body 100, a dental-tool protective casing 200, and a sterilizing means 300. The support body 100 includes a turntable 110 which is rotatably provided on an upper portion of the support body 100, and a power button 120 which is provided at a predetermined position on a surface of the support body 100 to control the supply of power. The dental-tool protective casing 200 is coupled at an open bottom thereof to the upper portion of the support body 100, with a door 210 provided on a side of the dental-tool protective casing 200. The sterilizing means 300 is provided in the dental-tool protective casing 200, and includes an ultraviolet lamp 310 that radiates ultraviolet rays when power is supplied to the sterilizing means 300. Since the above components are equal to those of the first to third embodiments, they will not be described below. A dental-tool holding means 400 of the fourth embodiment different from those of the first to third embodiments will be described below in detail.

In the dental-tool holding means 400 according to the fourth embodiment of the present invention, locking protrusions 460 are provided on a surface of the turntable 110 in such a way as to protrude upwards, thus making the dental tools DT be locked in a standing position. A dental tool DT which may be fitted over each locking protrusion 460 is a handpiece, etc. The dental-tool holding means 400 of the fourth embodiment is preferably applied to a long and large dental tool.

Meanwhile, the turntable 110 of the present invention preferably comprises a manual turntable 110 which is rotated only when a person artificially rotates the turntable 110. The dental-tool holding means 400 according to the fourth embodiment of the present invention is used to sterilize the dental tool DT, such as a handpiece, with the ultraviolet rays radiated from the ultraviolet lamp 310.

Further, according to the present invention, light reflecting films 230 are provided on an inner surface of the protective casing 200 and an inner surface of the door 210 to reflect light, radiated from the ultraviolet lamps 310 and 450, onto the dental tools DT, thus enhancing the power for sterilizing the dental tools DT. Further, a surface of each light reflecting film 230 is coated with a photo-catalyst 240 having a photo-catalytic function, so that the power for sterilizing the dental tools DT is further increased.

Further, only the photo-catalyst 240 may be applied to the inner surface of the protective casing 200 and the inner surface of the door 210 without the light reflecting film 230 so as to increase the power for sterilizing the dental tools DT. There are various kinds of photo-catalysts 240. However, it is most preferable to use titanium dioxide (TiO₂).

As described above, the present invention provides a dental tool sterilizer, which enables various kinds of dental tools DT for treating the teeth to be seated and then be sterilized by turning on an ultraviolet lamp for a preset time, and which is constructed so that the ultraviolet lamp is turned off so as to be harmless to humans when the sterilized dental tools are taken out of the sterilizer, and the ultraviolet lamp is turned on when the sterilizing operation is performed again.

1. A dental tool sterilizer, comprising:
a support body having a rotatable turntable on an upper portion thereof, a power button being provided at a predetermined position on the support body to control supply of power;
a dental-tool protective casing provided such that an open bottom thereof is placed on the upper portion of the support body, and having a door on a side thereof;
sterilizing means provided in the dental-tool protective casing, and having an ultraviolet lamp that radiates ultraviolet rays when power is supplied to the sterilizing means; and
dental-tool holding means provided on the turntable in such a way as to rotate along with the turntable, and holding a plurality of dental tools.

2. The dental tool sterilizer according to claim 1, wherein the power button, timer up/down buttons, a display part, an ultraviolet-lamp operating time setting button, and a display lamp are provided on a surface of the support body.

3. The dental tool sterilizer according to claim 1, wherein the door is a sliding door that conforms to a shape of the side of the dental-tool protective casing.

4. The dental tool sterilizer according to claim 1, wherein a storage part is provided on a central portion on an upper portion of the dental-tool protective casing, and functions to temporarily store a dental tool which is being used.

5. The dental tool sterilizer according to claim 1, wherein the sterilizing means comprises an ultraviolet lamp which is turned on or off in conjunction with operation of the power button.

6. The dental tool sterilizer according to claim 1, wherein a sensor is provided at a position adjacent to the door to control supply of power to the ultraviolet lamp.

7. The dental tool sterilizer according to claim 1, wherein the dental-tool holding means comprises:
support columns erected vertically and spaced apart from each other by predetermined intervals;
a support ring provided on each of upper and lower ends of the support columns;
a plurality of holders provided on each of the support columns or the support ring, and having a plurality of steps; and
a dental-tool holding ring provided on each of the steps, and having a dental-tool holding hole to hold each of the dental tools.

8. The dental tool sterilizer according to claim 7, wherein a portion of the dental-tool holding ring is cut to be open.

9. The dental tool sterilizer according to claim 1, wherein the dental-tool holding means comprises:
support columns erected vertically and spaced apart from each other by predetermined intervals;
a support ring provided on each of upper and lower ends of the support columns; and
a plurality of holders provided on each of the support columns or the support ring, each of the holders comprising:
a plurality of steps;
a light radiating space formed through a side surface of the holder; and
a dental-tool holding hole formed in each of the steps in such a way as to communicate with the light radiating space.

10. The dental tool sterilizer according to claim 1, wherein the dental-tool holding means comprises:
support columns erected vertically and spaced apart from each other by predetermined intervals;
a support ring provided on each of upper and lower ends of the support columns;
a tray support provided on each of the support columns or the support ring, and having a locking step; and
a tray coupled to the locking step, and receiving the dental tools.

11. The dental tool sterilizer according to claim 10, wherein a portion of the tray is cut to be open.

12. The dental tool sterilizer according to claim 1, wherein the dental-tool holding means comprises a locking protrusion which protrudes upwards so as to make each of the dental tools be locked to a surface of the turntable in a standing position.

13. The dental tool sterilizer according to claim 1, wherein an ultraviolet lamp is provided on a surface of the turntable.

14. The dental tool sterilizer according to claim 1, wherein an inner surface of each of the protective casing and the door is coated with a photo-catalyst.

15. The dental tool sterilizer according to claim 1, wherein a light reflecting film is provided on an inner surface of each of the protective casing and the door.

16. The dental tool sterilizer according to claim 15, wherein a surface of the light reflecting film is coated with a photo-catalyst.
